(19) United States
(12) Patent Application Publication
(10) Pub. No.: US 2026/0043793 A1
(43) Pub. Date: Feb. 12, 2026

(54) MICROFLUIDIC CELL SPHEROIDS AND ORGANOIDS CULTURE INSERT FOR DISEASE MODELING AND SCREENING OF THERAPEUTICS

(71) Applicant: APRICELL BIOTECHNOLOGY INC., Victoria (CA)

(72) Inventors: Amir SEYFOORI, Victoria, BC (CA); Mohsen AKBARI, Victoria, BC (CA)

(21) Appl. No.: 19/100,229

(22) PCT Filed: Aug. 3, 2023
(86) PCT No.: PCT/CA2023/051039

§ 371 Date: Jan. 31, 2025

Related U.S. Application Data
(60) Provisional application No. 63/394,882, filed on Aug. 3, 2022.

(51) Int. Cl. G01N 33/50; C12M 1/00; C12M 1/32; C12M 3/06

(52) U.S. Cl. CPC G01N 33/5088; C12M 23/12; C12M 23/16; C12M 23/34; C12M 29/00; G01N 2500/10

(57) ABSTRACT

A microfluidic device is provided for culturing cell spheroids or organoids and therapeutics screening, the microfluidic device comprising a shell and an at least one sector, the shell comprising a multiplicity of segments, each segment defined by a center point, an outer wall, and radially extending walls that radiate from the center point to the outer wall, the sector retained in the segment and comprising a plurality of microwells, a loading well which is in elevated relationship with the plurality of microwells, a plurality of micro-troughs which extend between the loading well and the plurality of microwells, such that each microwell is in fluid communication with the loading well via a micro-trough, a delivery port, and a plurality of delivery troughs which extend between the delivery port and the plurality of microwells such that each microwell is in fluid communication with the delivery port via a delivery trough.

### FIELD

The present technology relates to an insert for a well of a multi-well plate to which cells, hydrogels and culture medium can easily be added to multiple microwells. More specifically, it is an integrated device for delivering one or more therapeutics or other additives under temporal control to individual microwells of the device.

### BACKGROUND

Despite advances in the treatment of cancer patients, the burden of cancer deaths remains high, with 11 million cancer deaths reported worldwide in 2020. A key limitation in cancer treatment is the uncertainty of oncologists in predicting the treatment response for individual patients, resulting in patients receiving ineffective treatment with unnecessary exposure to toxic side effects and high treatment costs. On the other hand, effective predictive biomarkers are needed to enable personalized medicine and increase survival for cancer patients. Personalized medicine strategies in oncology have been largely based on genomic biomarkers. However, less than half of patients are eligible for genetically matched treatment and for the majority of anticancer agents, no genetic markers are available. Hereby, patient-derived organoids (PDOs) have been recently introduced as an alternative approach in precision medicine and as a predictive biomarker for treatment response in cancer patients. PDOs have several advantages as a personalized treatment approach over the common two-dimensional (2-D) in vitro and animal-based models called patient-derived xenografts (PDX).

When cancer cells are cultured as 2-D monolayers, they cannot recreate the complex interactions between cancer cells and other tumor microenvironment compartments, leading to limited success in translating in vitro results to clinical use. This is due to the fact that they don't reflect the heterogeneity of the primary tumor, as well as tissue-specific architecture and mechanical stresses. On the other hand, tumor organoids can overcome these deficiencies. Compared to PDX models, PDOs can be established in a shorter period and are much more economical. PDX models require up to 6 months to establish tumor growth and retain complications from infiltrating murine stromal cells. Although tumor organoids enable recapitulating the complexity of the tissue microenvironment, they fail in the process of medium/high-throughput drug screening goals. Therefore, conventional methods for making tumor organoids cannot be used as a practical assistive tool to test the efficacy of cancer chemotherapeutics in humans. To investigate the efficacy of each chemotherapeutic agent in individual patients, developing an advanced PDO model for screening these interventions is crucial. Today, three-dimensional (3-D) tumor organoid models generated from the primary patients in combination with immune cells are used as a powerful alternative to the monolayer culture for testing the efficacy responses of either pre-FDA approval or post-FDA approval chemo/immunotherapeutic in different cancers. However, in the context of complex and human-mimicked tumor modelling, these organoid models face shortcomings, including 1) incomplete biomimicry of tumor cells interactions with stromal cells such as immune and cancer-associated fibroblast (CAF) cells and 2) lack of a proper vascular network that might affect drug transportation within the tumor.

Methods for producing suitable devices for culturing organoids are disclosed. For example, United States Patent Application Publication No. 20220062891 discloses a method and device for creating a lumen model in a microwell plate defining a well. A rod is inserted through the well of the microwell plate and the well is filled with a polymerizable material. The material is polymerized in the well. The rod is removed from the well of the microwell plate such that the polymerized material defines a lumen. This is a very simple and rudimentary device.

United States Patent Application Publication No. 20220042975 discloses methods of generating organoids on multi-well plates by depositing a polymeric solution comprising cells under conditions which result in a homogenous population of organoids, which can be used for high throughput analysis for drug screening and for determining treatment regimens of a drug.

United States Patent Application Publication No. 20170199173 discloses a microfluidic device that provides high throughput generation and analysis of defined three-dimensional cell spheroids with controlled geometry, size, and cell composition. The cell spheroids of the invention mimic tumor microenvironments, including pathophysiological gradients, cell composition, and heterogeneity of the tumor mass mimicking the resistance to drug penetration providing more realistic drug response. The device is used to test the effects of antitumor agents.

United States Patent Application Publication No. 20210123007 discloses a perfusion plate that can be combined with pillar plates containing cell layers. The perfusion plate can have an inflow reservoir and an outflow reservoir connected by at least one channel, which fluidly connects the perfusion wells to the reservoirs for the flow of a fluid such as growth media. A perfusion plate can be part of an assembly containing a pillar plate, a lid, and a transparent bottom for visualizing cell growth in the perfusion wells. The perfusion-pillar plate assembly can facilitate perfusion-based tissue culture and tissue communication for high throughput, high-content, drug screening.

United States Patent Application Publication No. 20200224137 discloses a method of producing uniformly sized organoids/multicellular spheroids using a microfluidic device having an array of microwells. The method involves several successive steps. First, a microfluidic device containing parallel rows of microwells that are connected with a supplying channel is filled with a wetting agent. The wetting agent is a liquid that is immiscible in water. For example, the wetting agent may be an organic liquid such as oil. In the next step, the agent in the supplying channel and the microwells is replaced with a suspension of cells in an aqueous solution that contains a precursor for a hydrogel. Next, the aqueous phase in the supplying channel is replaced with the agent, which leads to the formation of an array of droplets of cell suspension in the hydrogel precursor solution, which were compartmentalized in the wells. The droplets are then transformed into cell-laden hydrogels. Subsequently, the agent in the supplying channel is replaced with the cell culture medium continuously flowing through the microfluidic device and the cells within the hydrogels are transformed into multicellular spheroids.

What is needed is a multifunctional screening platform for tissue modeling and individualized chemotherapeutic or immunotherapeutic drug screening. This would preferably employ a screening platform enabling complex tissue modeling as well as medium/high throughput drug screening at the same time. It would be preferable if the screening platform allowed for loading culture medium and cells to multiple microwells in one step. It would be further preferable if other components of the tissue microenvironment such as extra-cellular matrix (ECM), and stroma could be applied at the same time to the same multiple microwells. It would be further preferable if one or more drugs or other compound could be added in a controlled manner, both with regard to timing and volume. It would be further preferable if the screening platform allowed for sequential treatment of the cells to a given number of drugs or other additives and alternatively allowed for combinatorial treatment of the cells to a given number of drugs or other additives. Still further, it would be preferable if the multiple cavities could be used to provide repetitions of a treatment such that the results could be statistically analyzed.

### SUMMARY

The present technology provides a multifunctional screening platform for tissue modeling and individualized chemotherapeutic or immunotherapeutic drug screening. The screening platform enabling complex tissue modeling as well as medium/high throughput drug screening at the same time. The screening platform allows for loading culture medium and cells to multiple cavities in one step. Other components of the tissue microenvironment such as ECM, and stroma can be applied at the same time to the same multiple microwells. One or more drugs or other compound can be added in a controlled manner, both with regard to timing and volume. The screening platform allows for sequential treatment of the cells to a given number of drugs or other additives and alternatively allows for combinatorial treatment of the cells to a given number of drugs or other additives. The multiple cavities can be used to provide repetitions of a treatment. In one embodiment, a microfluidic device is provided for use with a multi-well culture plate, for culturing cell spheroids or organoids for drug screening, the microfluidic device including at least one sector, each sector comprising: an outer edge; a plurality of microwells; a loading well which is in elevated relationship with the plurality of microwells; a plurality of micro-troughs which extend between the loading well and the plurality of microwells, such that each microwell is in fluid communication with the loading well via a micro-trough; a delivery port; and a plurality of delivery troughs which extend between the delivery port and the plurality of microwells such that each microwell is in fluid communication with the delivery port via a delivery trough.

The microfluidic device may further comprise a multiplicity of segments, each segment defined by a center point and radially extending walls that radiate from the center point to the outer wall, the sector retained in the segment.

In the microfluidic device, each segment may further comprise an inner wall disposed between the outer edge and the center point to define a second delivery port.

In the microfluidic device, each segment may further comprise a second delivery trough which extends between the second delivery port and the microwell such that the second delivery port and each microwell are in fluid communication.

In the microfluidic device, the sector may be comprised of a hydrogel.

In the microfluidic device, each segment may further comprise a pool which is disposed below the microwells.

In the microfluidic device, the sector may be comprised of a biocompatible rigid plastic polymer.

In the microfluidic device, the sector may be configured to be releasably retained in a well of a multi-well culture plate.

In the microfluidic device, the radially extending walls may be three-dimensionally printed in a well of the multi-well culture plate.

In another embodiment, a microfluidic device is provided for culturing cell spheroids or organoids and screening of therapeutics, the microfluidic device comprising a shell and at least one sector, the sector including at least one cell spheroids or organoid forming module and a first delivery module, the first delivery module in fluid communication with the cell spheroids or organoid forming module, the sector retained in the shell, the microfluidic device configured for retention in a well of a culture plate.

In the microfluidic device, the cell spheroids or organoid forming module may include a plurality of microwells, a loading well which is in elevated relationship with the plurality of microwells, a plurality of micro-troughs which extend between the loading well and the plurality of microwells, such that each microwell is in fluid communication with the loading well via a micro-trough.

In the microfluidic device, the first delivery module may include a delivery port, and a plurality of delivery troughs which extend between the delivery port and the plurality of microwells such that each microwell is in fluid communication with the delivery port via a delivery trough.

In the microfluidic device, the shell may include a second delivery module which is in fluid communication with the cell spheroids or organoid forming module.

In the microfluidic device, the second delivery module may include a second delivery port, and a delivery microchannel which extends between the second delivery port and the first delivery module such that the second deliver port is in fluid communication with the first delivery module.

In the microfluidic device, the sector may comprise at least one hydrogel.

The microfluidic device may further comprise a pool which is in indirect fluid communication with at least one microwell.

In the microfluidic device, the pool may be disposed below at least one microwell.

In another embodiment, a method of screening at least two therapeutics is provided, the method comprising: selecting a microfluidic device, the microfluidic device comprising at least one sector, the sector including at least one cell spheroids or organoid forming module and a first delivery module, the first delivery module in fluid communication with the cell spheroids or organoid forming module, the sector retained in the shell, the shell including a second delivery module, the second delivery module in fluid communication with the cell spheroids or organoid forming module; loading cells into the cell spheroids or organoid forming module; culturing the cells in the cell spheroids or organoid forming module to provide cell spheroids or organoids; loading a first therapeutic into the first delivery module; loading a second therapeutic into the second delivery module; and determining the effect on cell spheroids or organoid function.

In the method, the second therapeutic may be loaded concomitantly with the first therapeutic.

In the method, the second therapeutic may be loaded after the first therapeutic is loaded. The method may further comprise loading at least one ECM hydrogel into the either cell spheroids or organoid forming module or the first delivery module.

The method may further comprise loading a second ECM hydrogel into the first delivery module.

In another embodiment, a method of screening at least one therapeutic is provided, the method comprising: selecting the microfluidic device of claim 1; loading cells into the loading well; culturing the cells in the microwells to provide cell spheroids or organoids; loading a first therapeutic into the delivery port; and determining the effect on cell spheroids or organoid function.

The method may further comprise loading an ECM hydrogel into the loading well prior to loading the first therapeutic.

The method may further comprise loading a second therapeutic into the delivery port after the first therapeutic is loaded.

### FIGURES

FIG. 1 is a top perspective view of the microfluidic device of the present technology.

FIG. 2 is a top perspective view of an alternative embodiment microfluidic device.

FIG. 3 is a top perspective view of the microfluidic device of FIG. 2.

FIG. 4 is a cross sectional view of the microfluidic device of FIG. 1.

FIG. 5A is a cross sectional view of another alternative embodiment microfluidic device; and FIG. 5B is a top perspective view of the alternative embodiment microfluidic device.

FIG. 6 is a plan view of the insert of the microfluidic device of FIG. 1 in a culture plate.

FIG. 7 is a top perspective view of another alternative embodiment microfluidic device.

FIG. 8 is a top view of another alternative embodiment microfluidic device.

FIG. 9 is a cross section partial perspective view of the microfluidic device of FIG. 8.

FIG. 10 is a plan view of a 12 well-plate filled with the microfluidic devices.

FIG. 11 is a plan view of a 48 well plate filled with the microfluidic devices.

FIG. 12 is a plan view of a 96 well plate, showing that the wells can be filled with the microfluidic devices.

FIGS. 13A to 13H shows the results of glioblastoma U251 cell spheroids or organoids cultured in collagen ECM in the device or sector. FIG. 13A is at day zero of culturing; FIG. 13B is at day one of culturing; FIG. 13C is at day three of culturing; FIG. 13D shows the live-dead fluorescence image of the cell spheroids or organoids at day three of culturing; FIG. 13E shows live-dead fluorescence in the absence of the drug; FIG. 13F shows live-dead fluorescence at 250 μM of drug; FIG. 13G shows live-dead fluorescence at 500 μM of drug; and FIG. 8H shows a graph of invasion length versus drug concentration.

FIG. 14 is a graph showing cell viability of U251 glioblastoma cell spheroids or organoids in different Reelin concentrations.

FIGS. 15A to 15D shows the behavior of the Ovarian cancer SKOV-3 cell spheroids or organoids cultured in collagen ECM in the device or sector in the presence of the CAR-T cells; FIG. 15A is at day zero of culturing; FIG. 15B is at day 2 of culturing; FIGS. 15C & D show fluorescence live and dead images of the cell spheroids or organoids at day 3 of culturing respectively. FIGS. 15E to H shows the behavior of the ovarian cancer SKOV-3 cell spheroids or organoids cultured in collagen ECM in the device or sector in the absence of the CAR-T cells. FIG. 15E shows cell spheroids or organoid in the ECM gel is at day zero of culturing; FIG. 15F is at day 2 of culturing; FIGS. 15G&H show the fluorescence images of the cell spheroids or organoids at day 3 of culturing; and FIG. 15I is a graph showing the metabolic activities of cell spheroids or organoids cultured in the collagen in the device or sector after CAR-T cell therapy.

FIGS. 16A and 16B are graphs showing cell spheroids or organoids diameter versus Reelin concentration for non-resistant and resistant to Temozolomide (TMZ) cell spheroids or organoids respectively at day 0 and 3 of culturing in the device or sector; FIGS. 16 C and D show the invasion length of non-resistant and resistant to Temozolomide cell spheroids or organoids respectively within the collagen/HA ECM in the device or sector versus Reelin concentration; FIGS. 16 E and F show the invasion length non-resistant and resistant to Temozolomide cell spheroids or organoids respectively in Collagen/Reelin ECM versus TMZ concentration.

FIG. 17A shows the fluorescence images of the invaded non-resistant and resistant to Temozolomide u251 glioblastoma cell spheroids or organoids within the collagen/HA ECM hydrogel in the presence of the zero and 10 nM Reelin; and FIG. 17B is quantification of the invasiveness of the cell spheroids or organoids (resistant and non-resistant) within the collagen/HA ECM matrix at zero and 10 nM Reelin.

FIGS. 18A and 18B shows immunohistochemistry fluorescence images of the U251 glioblastoma cell spheroids or organoid slices in A) normaxia and B) hypoxia conditions stained with HIF-1α and DAPI; and FIG. 18C is a graph of fluorescent intensity of the HIF-1 α protein expression as a marker of hypoxic cells when treated with 50nM deferoxamine (DFO) in both normaxia and hypoxia conditions.

FIG. 19A shows the bright filed images of the Panc-1 spheroids co-cultured with human-derived fibroblasts cells in different ratios of 100%, 70%, 50% and 0%; and FIG. 19B is quantification of the tumor spheroid sizes with different co-culture conditions.

FIG. 20A shows the bright field invasion images of the Panc-1 spheroids co-cultured (co-seeded) with human-derived fibroblast cells in different ratios; and FIG. 20B is the quantification of the relative invasion length of the co-cultured tumor spheroids in the device or sector at day 1 and day 5.

FIG. 21A shows the bright field invasion images of the Panc-1 spheroids co-cultured with human-derived fibroblast cells in mixture with collagen ECM in the secondary delivery port of the sector from day 0 to day 5; and FIG. 21B is the quantification of the relative invasion length of the co-cultured spheroids within the ECM in different co-culture conditions.

FIG. 22A shows cell aggregates that were cultured in the rod shaped microwell; FIG. 22B shows cell aggregates that were cultured in the ring shaped microwell; and FIG. 22C shows cell aggregates that were cultured in the honeycomb shaped microwell.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### DEFINITIONS

Fluid communication—in the context of the present technology, fluid communication includes liquids, hydrogels, solutions and liquid mixtures that are capable of flowing or being injected.

Spheroid—in the context of the present technology a spheroid is a generally round collection of cells.

Organoid—in the context of the present technology an organoid is a group of cells that emulate an organ. Organoids may comprise one differentiated cell type, or two or more differentiated cell types, depending upon the particular tissue or organ being modeled or emulated.

Cell spheroids or organoid—in the context of the present technology, a cell spheroids or organoid is a tumor-like organoid. Cell spheroids organoids typically derive from primary tumors and can mimic the tumor microenvironment.

Biocompatible—in the context of the present technology biocompatible refers to any product which when in contact with cells, tissues or body fluid of an organism does not induce adverse effects such as immunological reactions and/or rejections, cellular death and the like. It will be appreciated that a biocompatible product can also be a biodegradable polymer.

Rigid—in the context of the present technology, a rigid plastic polymer is one that has a shape that once formed, remains constant. In contrast, a malleable plastic polymer is one whose shape can be varied.

### DETAILED DESCRIPTION

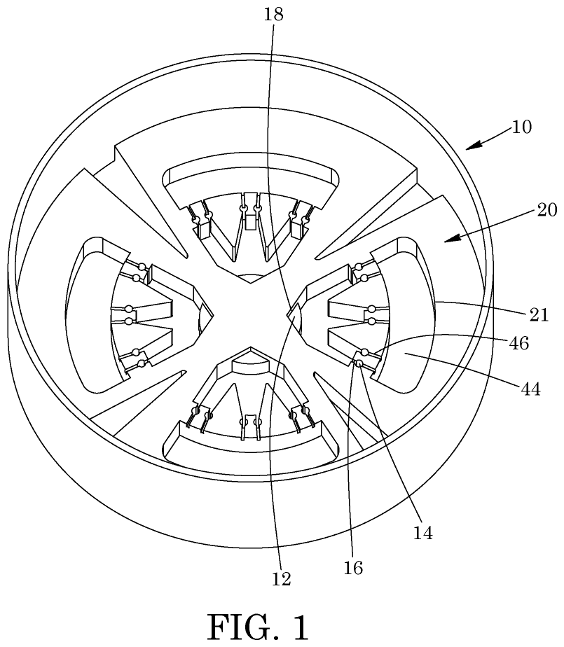

A microfluidic device, generally referred to as 10 is shown in FIG. 1. It has a number of sectors 20, separated by a V-shaped void which extends out radially between the sections. Each sector 20 has a number of loading wells 12 each which is in fluid communication with at least a plurality of microwells 14 via at least a plurality of micro-troughs 16, which slope downward on a consistent slope from the loading well 12 to the microwells 14. The loading well 12 is located proximate to the apex 18 of the sector 20, hence the micro-troughs 16 extend radially outward to the microwells 14. Each sector 20 is bounded by a wall 21 that extends upward and around the perimeter of the sector 20. A first delivery port 44 is in fluid communication with each microwell 14 via a delivery trough 46.

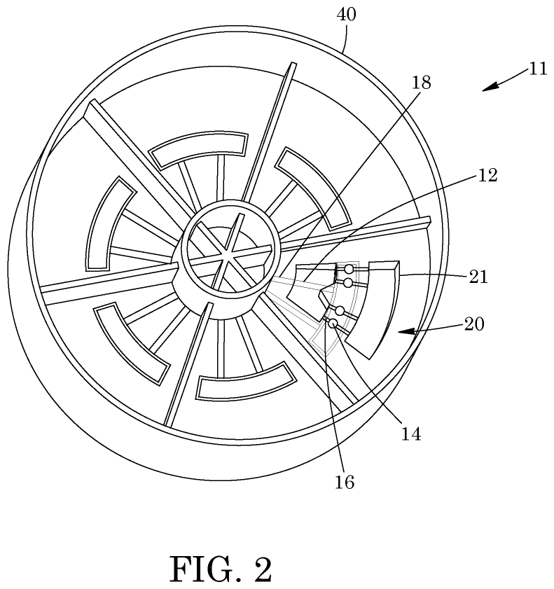

In an alternative embodiment shown in FIG. 2, the microfluidic device 11 includes an outer shell 40 and a multiplicity of sectors 20. Each sector 20 has a number of loading wells 12 each which is in fluid communication with a at least a plurality of microwells 14 via at least a plurality of micro-troughs 16, which slope downward on a consistent slope from the loading well 12 to the microwells 14. The loading well 12 is located at the curved apex 18 of a sector 20, hence the micro-troughs 16 extend radially outward to the microwells 14. Each sector 20 is bounded by a wall 21 that extends upward and around the perimeter of the sector 20.

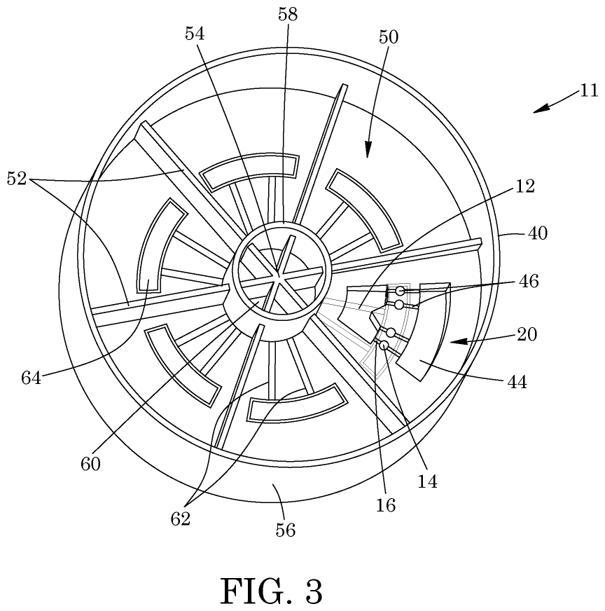

As shown in FIG. 3, in the microfluidic device 11 of FIG. 2, the sectors 20 each include the loading well 12, the multiplicity of microwells 14, the multiplicity of micro-troughs 16, a first delivery port 44 and a first delivery trough 46 for each microwell 14. The first delivery port 44 is in fluid communication with the microwells 14 via the first delivery troughs 46. The outer shell 40 consists of a rigid plastic polymer, for example, but not limited to polystyrene, polycarbonate, polyvinyl chloride, polymethyl methacrylate, 3-dimensional printed resin or any other gamma radiation stable plastic. It is biocompatible.

The outer shell 40 is divided into segments, generally referred to as 50 by radially extending walls 52 that radiate from a center point 54 to an outer wall 56. An inner wall 58, the radially extending walls and the center point 54 define a second delivery port 60 in each segment 50. Extending from each second delivery port 60 are at least two delivery microchannels or troughs 62 which terminate in a pool 64. The pool 64 sits about 500 μm or less below the microwells 14, when the sector 20 is in place. There is direct fluid communication between the second delivery port 60 and the pool 64, and indirect fluid communication between the pool 64 and the microwells 14 as the drug diffuses through the hydrogel of the sector 20.

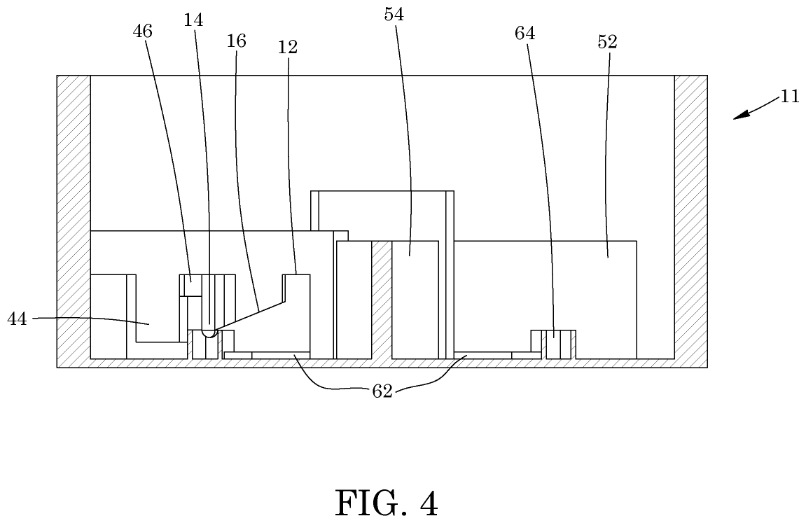

As shown in FIG. 4, the loading well 12 is elevated from the microwells 14.

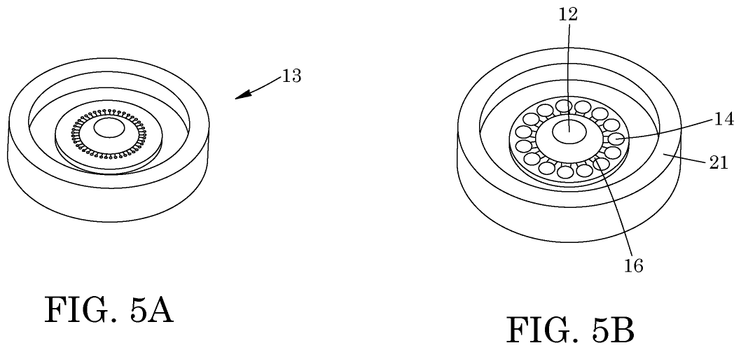

In another alternative embodiment shown in FIGS. 5A and B, the microfluidic device, generally referred to as 13 has a central loading well 12 which is in fluid communication with at least a plurality of microwells 14 via at least a plurality of micro-troughs 16, which slope downward on a consistent slope from the loading well 12 to the microwells 14. The loading well 12 is centrally located, hence the micro-troughs 16 extend radially outward to the microwells 14. The ring of microwells is bounded by a wall 21 that extends upward and around the perimeter of the microfluidic device 13.

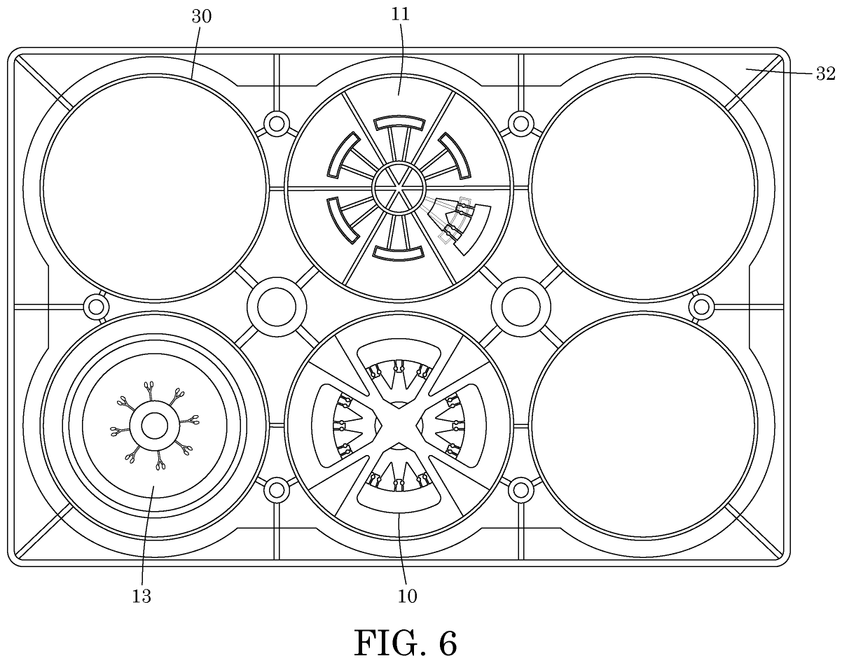

As shown in FIG. 6, the microfluidic device 10, 11, 13 is sized and shaped to fit in the well 30 of a microtiter plate 32 or culture plate. In the preferred embodiment, the microtiter plate 32 has between 6 and 98 wells. The loading wells 12 and the sloped micro-troughs 16 allow for loading of cells in culture medium into the multiplicity of microwells 14 in a given sector 20 or sector 20 in one step. The cells in culture medium flow under the force of gravity into the microwells 14. The microwells 14 can be different diameters (100-4000 μm) and different shapes, for example conical or pyramidal.

In one embodiment the sectors 20 and the device 10, 13 consist of, for example, but not limited to Polydimethylsiloxane (PDMS) or a hydrogel selected from synthetic polymeric hydrogels or natural polymeric hydrogels which are, for example, but not limited to alginate, agarose, polyethylene glycol (PEG), PEG-based hydrogels, polysaccharide hydrogels, gelatin-derived materials such as gelatin methacryloyl, cellulose-based hydrogels such as ethyl cellulose, methyl cellulose and cellulose acetate, and poly (N-isopropylacrylamide) and its derivate and copolymers with PEG, and gelatin. The hydrogel is biocompatible. In one embodiment, the sectors 20 include a combination of natural polymers and a combination of synthetic polymers.

The device 10, 11, 13 is used for in-vitro and ex-vivo generation of tissue-mimicked organoids or cell spheroids, in addition to testing of therapeutics. Cell spheroids or organoid formation occurs inside the microwell 14 while drug delivery can occur through the delivery troughs 46, 62 from the delivery ports 44,60 allowing for the potential to treat the cell spheroids or organoids with one or a combination of drugs simultaneously or sequentially. The drug formulation includes but is not limited to free drugs, controlled-release formulations (lipid-based and polymeric-based), drug-loaded nanoparticles and drugs-loaded hydrogels. Moreover, the delivery port 44 has multiple applications for localized therapeutic delivery. This includes the insertion of drug-eluted meshes, injection of drug-encapsulated gels, and direct printing of the drug-eluted construct within the delivery port. Additionally, extra-cellular matrix hydrogels, for example, but not limited to collagen, hyaluronic acid, Matrigel®, alginate, gelatin, fibrin/fibrinogen, silk fibroin, chitosan and laminin can be added to the microwells 14 via the loading wells 12 or via the delivery port 44. Similarly, immune cells can be added to the microwells 14 via the delivery ports 44, 60 or the loading wells 12. Hydrogels carrying endothelial cells can also be loaded into one of the first delivery ports 44 or the loading wells 12, allowing for vascularization of the spheroids/cell spheroids or organoids.

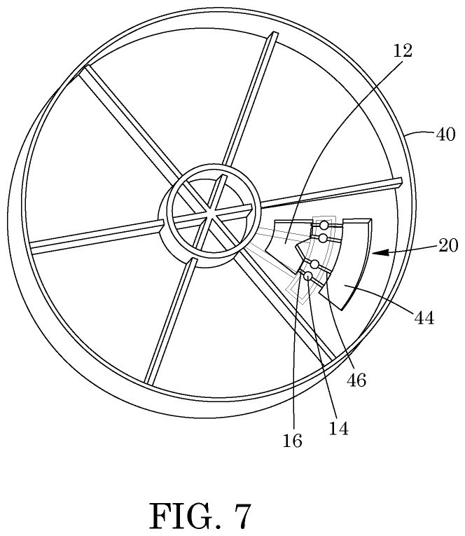

As shown in FIG. 7, in an alternative embodiment, both the outer shell 40 and the sector 20 consist of a rigid plastic polymer, for example, but not limited to polystyrene, polycarbonate, polyvinyl chloride, 3-dimensional printed resin or any other gamma radiation stable plastic. It is biocompatible.

In this embodiment, the loading well 12, and the multiplicity of micro-troughs 16 are used to deliver a different drug or compound to the microwell 14 than is delivered via the first delivery port 44 and the delivery troughs 46. Alternatively, in this embodiment, the different drug or compound can be delivered via the first delivery port 44 and the delivery troughs 46. Additionally, ECM hydrogels, for example, but not limited to collagen, hyaluronic acid, matrigel, alginate, gelatin, fibrin/fibrinogen, chitosan, fibrin, thrombin, silk fibroin and laminin can be added to the microwells 14 via the loading wells 12 or via the first delivery port 44. Similarly, secondary cells, such as, but not limited to fibroblasts, endothelial cells, immune cells, neural cells, astrocytes and microglial cells associated with the stroma of different cancerous and non-cancerous tissues can be added to the microwells 14 via the first delivery port 44 or the loading wells 12. Secondary cells can be also mixed with ECM hydrogels with desired ratios and be added to the microwells 14 via the loading wells 12 or first delivery port 44. Hydrogels carrying endothelial cells promote vascularization of the cell spheroids or organoids.

In another embodiment, the sector 20 consists of a malleable plastic polymer.

In all embodiments, the sector 20 is preferably releasably retained in the shell 40 and the device 10 is preferably releasably retained in a well 30 of a culture plate 32.

In use, cells (cell lines or patient-derived cells primary or stem cells) are deposited onto the loading wells 12, where they are distributed in the microwells 14 by gravity via the micro-troughs 16. The spheroids or organoids can be formed by one step seeding of single or multiple cells for mono-or co-or tri-culturing applications. The cells in each microwell 14 self-assemble into cell spheroids or organoids in 2-5 days. At specific time points, the cell spheroids or organoids in each microwell 14 can be individually treated with one or a combination of drugs simultaneously or sequentially. The drug formulation includes but is not limited to free drugs, controlled-release formulations (lipid-based and polymeric-based), and drugs-loaded hydrogels. ECM-associated hydrogels with/without additive stroma associated cells can be added to encapsulate the cell spheroids or organoids inside the hydrogel. Hydrogels carrying endothelial cells can also be loaded allowing for vascularization of the spheroids/cell spheroids or organoids. The health, viability and function of the cell spheroids or organoids is assessed after treatment with the drugs or any other therapeutic agents.

In one embodiment, the cell spheroids or organoid culture device 10, 13 was fabricated using replica molding of a stereolithography 3-dimensional printed construct in agarose hydrogel. High resolution photo curable resin was used for 3-dimensional printing of the mold with the layer thickness of 5 μm-25 μm. The 3-dimensional printed mold was washed with isopropyl alcohol and used for agarose replication and making the culture insert. An agarose solution with concentration of 1%-5% was cast on to the mold at 60° C. and removed from the mold after gelation at room temperature. The device 10, 13 was sterilized by exposing it to ultraviolet light with the maximum wavelength of 365 nm for 2 hours and placed into a well 30 of a 6 well plate 32 for further cell spheroids or organoid culture process.

In another embodiment, the sectors 20 were fabricated using replica molding of a stereolithography 3-dimensional printed construct in agarose hydrogel. High resolution photo curable resin was used for 3-dimensional printing of the mold with the layer thickness of 5 μm-25 μm. The 3-dimensional printed mold was washed with isopropyl alcohol and used for agarose replication and making the culture insert. An agarose solution with concentration of 1%-5% was cast on to the mold at 60° C. and removed from the mold after gelation at room temperature. The sectors 20 were sterilized by exposing it to ultraviolet light with the maximum wavelength of 365 nm for 2 hours and placed into an outer shell 40, which was then placed in a well 30 of a 6 well plate 32 for further cell spheroids or organoid culture process.

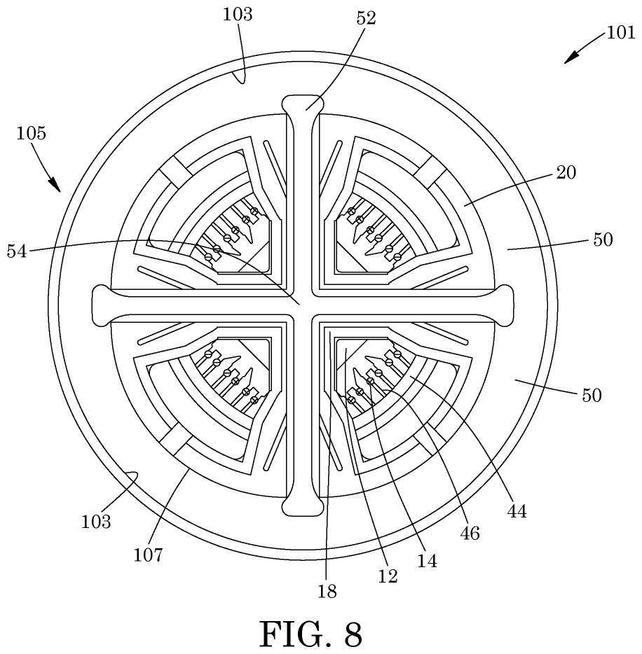

Another alternative embodiment of the microfluidic device, generally referred to as 101, is shown in FIG. 8. The segments 50 are separated by radially extending walls 52 that radiate from a center point 54 to an outer wall 103 of a well, generally referred to as 105. The radially extending walls 52 are 3-dimensionally printed inside the well 105. The sectors 20 are then placed in each segment 50. As shown in FIGS. 3 and 4, each sector 20 has a number of loading wells 12 each which is in fluid communication with a at least a plurality of microwells 14 via at least a plurality of micro-troughs 16, which slope downward on a consistent slope from the loading well 12 to the microwells 14. The loading well 12 is located at the curved apex 18 of a sector 20, hence the micro-troughs 16 extend radially outward to the microwells 14. Each sector 20 has an outer edge 107 that is bounded by the outer wall 103 of the well 105. The outer wall 103 of the well 105 extends upward and around the perimeter of the sector 20.

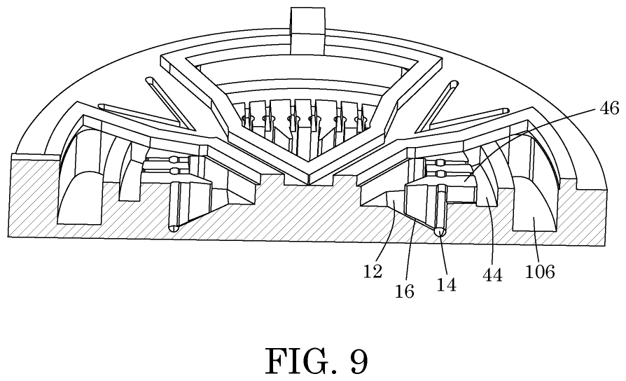

As shown in FIG. 9, the sectors 20 each include the loading well 12, the multiplicity of microwells 14, the multiplicity of first micro-troughs 16, a first delivery port 44 and a delivery trough 46 for each microwell 14. The first delivery port 44 is in fluid communication with the microwells 14 via the delivery troughs 46. A reservoir 106 is located in the sector 20.

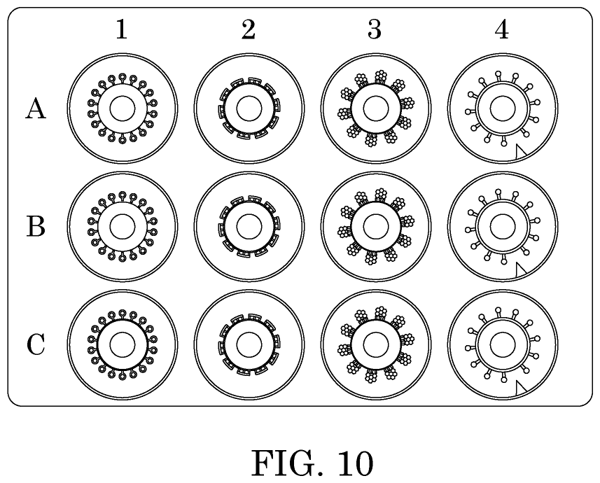

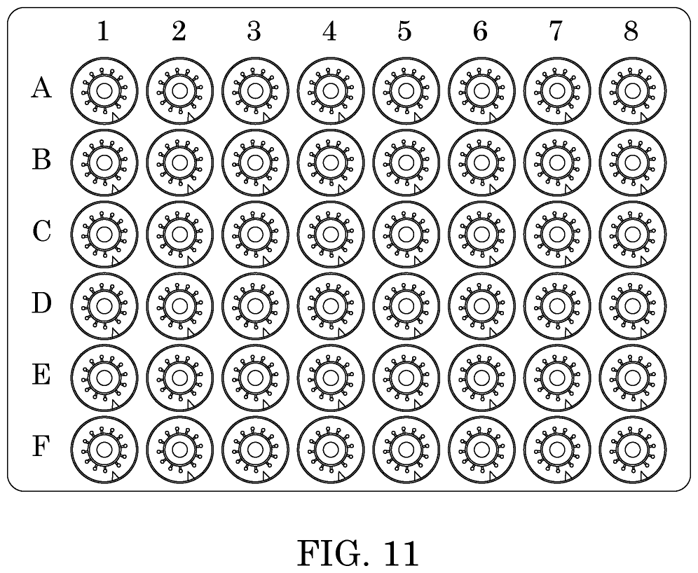

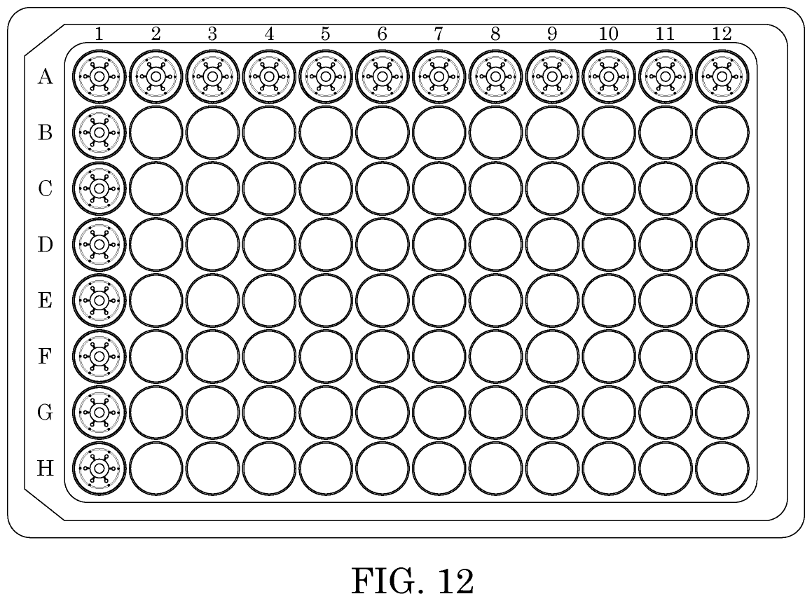

FIG. 10 shows a 12 well-plate 110 filled with the microfluidic devices 101. In one embodiment, the microwells are rod-shaped. In another embodiment, the microwells are a honeycomb shape. In another embodiment, the microwells are ring-shaped. FIG. 11 is a plan view of a 48 well cell culture plate 112 filled with the microfluidic devices 101. FIG. 12 is a plan view of a 96 well cell culture plate 114, showing that a 96 well cell culture plate 114 can be filled with the microfluidic devices 101.

### BENEFITS OF THE DEVICE

Presence of both cell spheroids or organoid forming module (loading wells 12, micro-troughs 15 and microwells 14) and first delivery module (first delivery port 44 and delivery troughs 46, and optionally second delivery port 60, delivery microchannels 62 and pool 64) in the same device 10, 11.
Presence of first delivery port 44 and delivery troughs 46, and optionally second delivery port 60, delivery microchannels 62 and pool 64 allows for addressing single or plurality of cell spheroids organoids with a certain drug without cross-contamination between adjacent microwells 14.
Presence of radially extending walls 52 defining segments 50 allows for addressing single or plurality of cell spheroids or organoids with a certain drug without cross-contamination between adjacent sectors 20.
Multiple drug delivery at the same time to the individual or plurality of cell spheroids or organoids using one or more of the first delivery port 44 and delivery troughs 46, and optionally second delivery port 60, delivery microchannels 62 and 64 and diffusion.
Presence of loading wells 12 and the sloped micro-troughs 16 allow for loading of cells in culture medium into the multiplicity of microwells 14 in a given sector 20 in one step.
Presence of loading wells 12 and extending walls reduces or eliminates cell waste allowing working with low cell number suitable for patient biopsy samples.
Presence of the first delivery port 44 and delivery troughs 46, and optionally second delivery port 60, delivery microchannels 62 and pool 64.
Compatibility of the device 10 with 3-dimensional extrusion printing.
Compatibility of the device 10 with a wide range of materials including naturally derived polymers and hydrogels (for example, but not limited to alginate, chitosan, agarose, gelatin and its derivate, collagen and its derivatives, hyaluronic acid, polyethylene glycol, and its derivates, cellulose-based hydrogels such as ethyl cellulose, methyl cellulose and cellulose acetate and its derivates. and synthetic polymers (for example, but not limited to polycaprolactone, polyester, poly lactic-co-glycolic acid, polydimethylsiloxane, polymethyl methacrylate, polyvinyl alcohol, poly(N-isopropylacrylamide). One or more natural polymer, hydrogel or synthetic polymer may be used.
The ability to produce cell spheroids and organoids with excellent size uniformity and reproducibility.
The ability to recapitulate the complexities of tumor microenvironment, including tumor-associated stroma, vasculature, and immune system in a high-throughput fashion.
Compatibility of the device with conventional immunofluorescent imaging and immunostaining analysis.
Compatibility of the device (due to using hydrogel-based material for the insert) with immunohistochemistry and tissue slicing protocols.
The ability of tumor tissue supernatant removing (due to open-well design of the insert) for downstream proteomics and cytokine analysis.
The ability of cell lysis on chip for extracting RNA and cell proteins and downstream transcriptomic and western blot analysis.
The ability to collect data from a sufficient number of replicates in the same device to allow for statistical analysis.

The Following Examples Were Conducted in the Device 11.

### Example 1

Glioblastoma Cell Spheroids or Organoid Formation Using U251 Cell Lines Glioblastoma cell lines, U251 were cultured in a culture medium consisting of Dulbecco's Modified Eagle Medium (DMEM) supplemented with 10% fetal bovine serum (FBS), and 100 mg/ml streptomycin. The U251 cells were incubated at 37° C. in a humidified atmosphere of 5% CO2, and at 90% confluency, were trypsinized into a single cell suspension. The cell suspension was centrifuged at 300 rpm for 5 minutes to avoid dead cell sedimentation. After removing the supernatant, cells were suspended in 1 ml of medium and counted using a standard hemocytometer. To generate U251 cell spheroids or organoids, cell suspension with the densities of ranging from 2.5×10⁵−5×10⁵ cells was prepared in 80 μL of the culture medium and was applied gently through the loading well 12 of the device 10 or sector 20. The loaded device 10, 11, 13 or sector 20 was kept in an incubator for 10 minutes to let the cells fill the microwells 14 through the micro-troughs 16 of the device 10 or sector 20. Afterwards, the culture medium in the loading well 12 was aspirated and was exchanged with a new medium. 200 μL of the new medium was gently added to the seeded cell in the microwells 14 through the first delivery port 44. U251 cell spheroids or organoids were monitored every day to measure their growth over the 4 days. In an alternative method, the new medium was added to the microwells 14 through the loading well 12. This sometimes led to loss of some cells from the microwell 14.

### Invasion Study of the U251 Cell Spheroids or Organoids on the Device or Sector in the Presence of Collagen/Hyaluronic Acid Hydrogel ECM

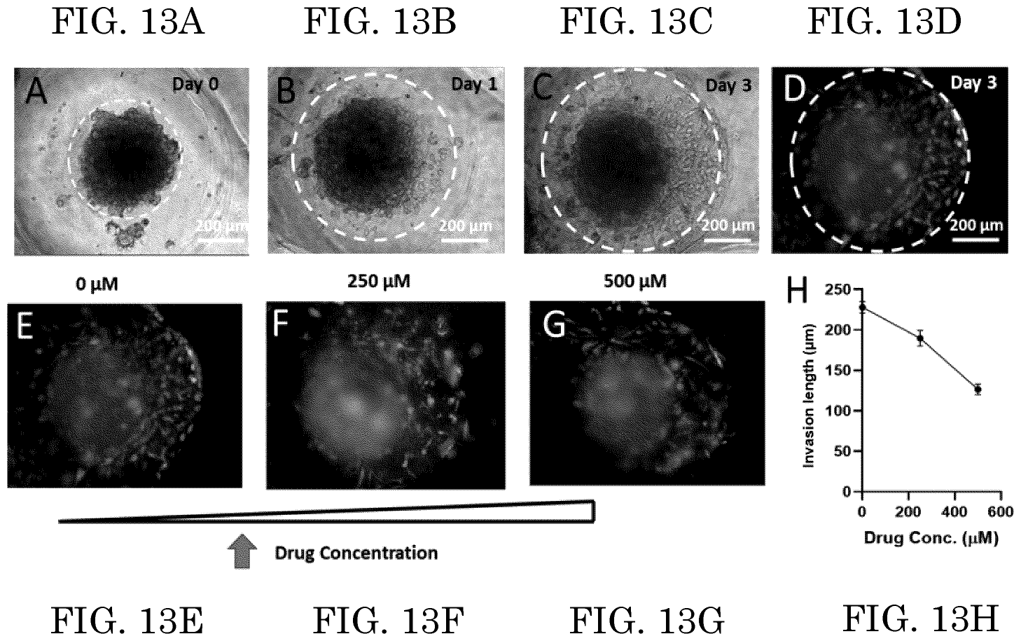

To investigate the invasiveness of the cell spheroids or organoids on the device 10 or the sector 20, the cell spheroids or organoids were encapsulated in a collagen/hyaluronic acid hydrogel. The hydrogel formulation mimics the extra cellular matrix of the brain tumor for better recapitulating of the tumor interaction with the environment. 50 μL of a hydrogel solution was gently pipetted through the loading well 12 of the device 10, 11, 13 or sector 20. It flowed through the micro-troughs 16 and filled the microwells 14 which retained the U251 cells. ECM hydrogel was formed after incubating at 37° C. for 30 hours. FIGS. 13A-D showing growth and invasion of the cell spheroids or organoid in ECM hydrogel prior to drug testing. FIG. 13E shows live-dead fluorescence in the absence of the drug. FIG. 13F shows live-dead fluorescence at 250 μM of drug. FIG. 13G shows live-dead fluorescence at 500 μM of drug. FIG. 8H shows a graph of invasion length versus drug concentration.

### Live-dead Staining of U251 Cell Spheroids or Organoids

To assess the viability of the tumor cells within the U251 cell spheroids or organoids during their formation and invasion within the ECM hydrogel and to observe the of invasion length and pattern of the tumor cell to the adjacent ECM in 4 days, Live/Dead assays were conducted using 1 μM calcein AM and 4 μM ethidium homodimer-1 (Life Technologies kit) for 30 minutes at 37° C. The whole cell spheroids or organoid staining and imaging process was conducted in the microwell 14.

### Drug Testing Against U251 Cell Spheroids or Organoids

Drug testing was performed on Cell spheroids or organoid in culture insert using the Temozolomide (TMZ) cancer chemotherapy drug at concentrations of 250 and 500 μM in DMEM medium to provide a drug solution. 100 μL of the drug solution was added to the first delivery port 44 of the device 10 or sector 20 and was delivered to the cell spheroids or organoids through the delivery troughs 46. 4 day-old spheroids were treated with drug by removing the primary culture medium followed by adding the drug solution and culturing for 3 days. The effect of drug treatment on the invasion length and viability of the cell spheroids or organoids were assessed using florescent microscopy and presto blue analysis respectively. The results are shown in FIGS. 8E-H. Drug treatment resulted in decrease in invasion length of the tumor cells within the ECM hydrogel inside the microwells 14. Increasing the drug concentration inhibited growth and invasion of the tumor cells within the ECM hydrogel.

### Example 2

### Ovarian Cancer Cell Spheroids or Organoid Formation in the Device or Sector Using Human-Derived SKOV-3 Cell Lines

Ovarian cancer cell lines, SKOV-3 were cultured in McCoy supplemented with 10% FBS, and 100 mg/ml streptomycin. The SKOV-3 cells were incubated at 37° C. in a humidified atmosphere enriched to have 5% CO2, and at 90% confluency, were trypsinized into a suspension of single cells. The cell suspension was centrifuged at 280 rpm for 5 minutes to avoid dead cell sedimentation. After removing the supernatant, cells were suspended in 1 ml of culture medium and counted using a standard hemocytometer. To generate SKOV-3 cell spheroids or organoids, cell suspensions with the densities of ranging from 2.5×10⁵−5×10⁵ cells were prepared in 80 μL of the culture medium and were applied gently through the loading well 12 of the device 10 or sector 20. The device 10, 11, 13 or the sector 20 was kept in an incubator for 10 minutes to allow the cells and culture medium to fill the microwells 14 through the micro-troughs 16. Afterwards, the culture medium in the loading well 12 was aspirated and is exchanged with a new medium. 200 μL of the new medium was gently added to the seeded cell in the microwells 14 through the first delivery port 44. SKOV-3 cell spheroids or organoids were monitored every day to measure their growth over the 6 days. In an alternative method, the new medium was added to the microwells 14 through the loading well 12. This sometimes led to loss of some cells from the microwell 14.

### Live-dead Staining of the Skov-3 Cell Lines

To assess the viability of the tumor cells within the SKOV-3 cell spheroids or organoids during their formation in 6 days, a Live/Dead assay was conducted using 1 μM calcein AM and 4 μM ethidium homodimer-1 (Life Technologies kit) for 30 minutes at 37° C. The whole cell spheroids or organoid staining and imaging process was conducted in the microwell 14.

### CAR-T (Chimeric Antigen Receptor-T) Cell Applying to the Cell Spheroids or Organoid Model

Immunotherapy of solid tumors has been less successful because immunosuppressive barriers impede immune cell trafficking and function against cancer cells. Current immuno-cytotoxicity assays in the preclinical efforts are limited to the monolayer co-culture of the tumor cells with cytotoxic lymphocytes in which the barrier effect of TME on immune cell function are not properly considered.

There is an unmet need for a bioengineered ex-vivo model of solid tumor enabling predicting the dynamic behavior of the immune cells interaction with the tumor stroma and extra cellular matrix (ECM).

To investigate the invasiveness of the SKOV-3 cell spheroids or organoids on the device 10 or the sector 20, the cell spheroids or organoids were encapsulated in a collagen hydrogel. The hydrogel formulation mimics the extra cellular matrix of the ovarian tumor for better recapitulating of the tumor interaction with the environment. 50 μL of a hydrogel solution was gently pipetted through the loading well 12 of the device 10, 11, 13 or sector 20. It flowed through the micro-troughs 16 and filled the microwells 14 which retained the SKOV-3 cells. ECM hydrogel was formed after incubating at 37° C. for 30 hours.

To demonstrate the compatibility of the device 10 or the sector 20 with immune cell therapy efficacy testing, CAR-T cell suspension in Cell Therapy System (CTS) OpTmizer™ T-Cell Expansion media+Interleukin-2 with the ratio of 5:1 to tumor cells was applied through the first delivery port 44 of the device 10, 11, 13 or the sector 20. Immune cells were delivered to the cell spheroids or organoids in microwells 14 through the micro-troughs 16.

### Viability and Metabolic Activity Analysis of the Tumor Spheroids or Organoids After CAR-T Cell Treatment

The cytotoxicity efficacy of the Folate Receptor-a CAR-T against SKOV-3 ovarian cell spheroids or organoids was assessed using Live/dead and presto Blue analysis.

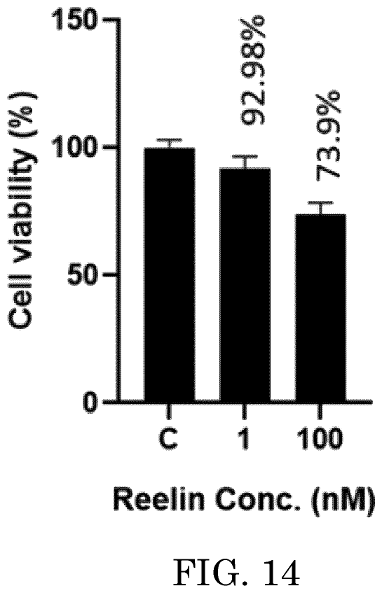

As shown in FIGS. 14A-H, presence of the CAR-T cells within the tumor microenvironment of the in-vitro 3D ovarian model has a significant effect on the invasion inhibition of the tumor cells within the ECM hydrogel in the microwell 14. Moreover, the number of dead tumor cells as a result of CAR-T cell toxicity was increased after applying immune cells to the model. In that condition, FIGS. 14A-D, tumor cells in the ECM hydrogel show more round shape structure with smaller surface area than the elongated cells in the control condition. As shown in FIG. 14I, metabolic activity of the cell spheroids or organoids in different experimental setting shows the effect of CAR-T cells on inhibiting the viability of the of SKOV-3 cells so that direct applying of the CAR-T cells is more toxic than the condition for presence of the CAR-T cells within the ECM hydrogel (Collagen). It demonstrates the inhibitory effect of ECM on function and efficacy of CAR-T cell therapy which can mimic the real in-vivo conditions. SKOV-3 cell spheroids or organoids as control positive and dimethyl sulphoxide (DMSO) treated cell spheroids or organoids as control negative were selected to approve the metabolic activity of the live and dead cells.

### Example 3

### Glioblastoma (GBM) Cell Spheroids or Organoid Formation in the Device or Sector Using U251 Cells Resistant and Non-Resistant to the Temozlomid (TMZ) Chemotherapy Drug

To investigate and compare the invasive behavior of TMZ resistant GBM cancer cells with non-resistant cell spheroids or organoids through the ECM hydrogel, we used TMZ resistant and non-resistant U251 cell lines to make cell spheroids or organoids in the device 10 or the sector 20. The method of U251 cell spheroids or organoid formation was similar to that disclosed above.

Invasion study of the U251 Cell Spheroids or Organoids in the Presence of Collagen/HA Hydrogel ECM Conditioned with Reelin Using Vimentin Staining

The effect of recombinant Reelin on viability of the U251 cell spheroids or organoids was studied. It was tested with 2 different concentrations of Reelin (1 and 100 nM) on cell spheroids or organoids in the device 10 or the sector 20. Moreover, to study the invasive behavior of the resistant and non-resistant cell spheroids or organoids, they were embedded in Collagen/HA hydrogel. The method of cell spheroids or organoid embedding in the hydrogel is similar to that disclosed in example 1. In this study, the effect of Reelin protein in combination with ECM hydrogel on invasiveness of the cell spheroids or organoids and their invasion length using Vimentin immunostaining and florescent microscopy respectively was studied.

As shown in FIG. 14, investigation of Reelin concentration on viability of the U251 cell spheroids or organoids shows a significant decrease in cell viability by increasing the Reelin concentration from 1 to 100 nM. It is shown in live-dead fluorescent images by increasing the number of red cells stained with Propodeum iodide (PI) compared to the green cells stained with calcein AM after treatment with 100 nM Reelin.

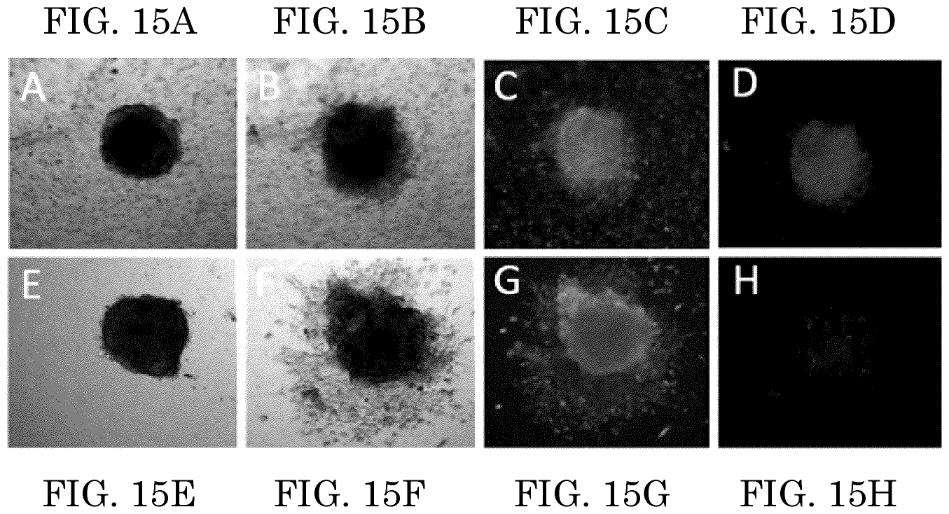

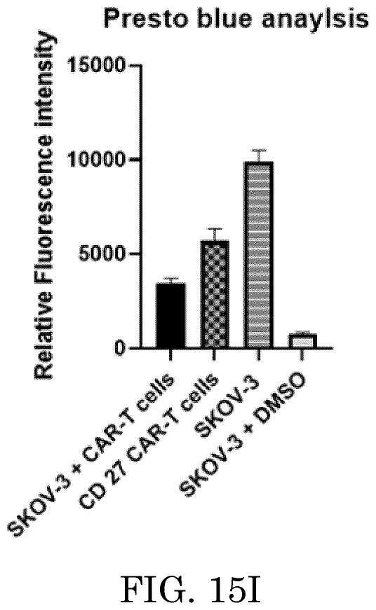

FIGS. 15A and 15B shows the effect of different Reelin concentrations on cell spheroids or organoids diameter with 2 different conditions of Temozolomide non-resistant and resistant cells respectively. As shown in FIG. 15B, the amount of size increase in non-resistant cell spheroids or organoids over time is more significant at each concentration of the Reelin in comparison to the resistant cell spheroids or organoids shown in FIG. 15B. However, higher concentrations of Reelin have more inhibitory effect on the growth of cell spheroids or organoids at day 3. This effect is less for resistant cell spheroids or organoids at day 3.

As depicted in FIGS. 15C and 15D invasion behavior of non-resistant cell spheroids or organoids is different from their growth behavior in response to the increasing concentrations of Reelin so that invasion length of the cell spheroids or organoids embedded inside the Collagen/HA matrix is constantly increasing. This behavior is also different for resistant cell spheroids or organoids, so that average invasion length rises to around 400 μm at 10 nM Reelin concentration and it drops significantly at 100 nM.

FIGS. 15E and 15F also show the invasion length of non-resistant and resistant cell spheroids or organoids embedded in hydrogel ECM matrix with 0 and 10 nM Reelin concentration in response to increasing amount of TMZ as a co-drug treatment approach. Co-treatment of TMZ in Reelin conditioned matrix of each cell spheroids or organoids shows the significant decrease in the length of invasion by increasing amounts of TMZ drug. However, cell spheroids or organoids embedded in Reelin treated matrix present higher invasion length compared to the normal ECM matrix in response to TMZ. This condition is reverse in the case of treating the resistant cells embedded within the normal and Reelin conditioned matrix. FIGS. 15G&H show the fluorescence images of the cell spheroids or organoids at day 3 of culturing. FIG. 15I is a graph showing the metabolic activities of cell spheroids or organoids cultured in the collagen in the device or sector after CAR-T cell therapy.

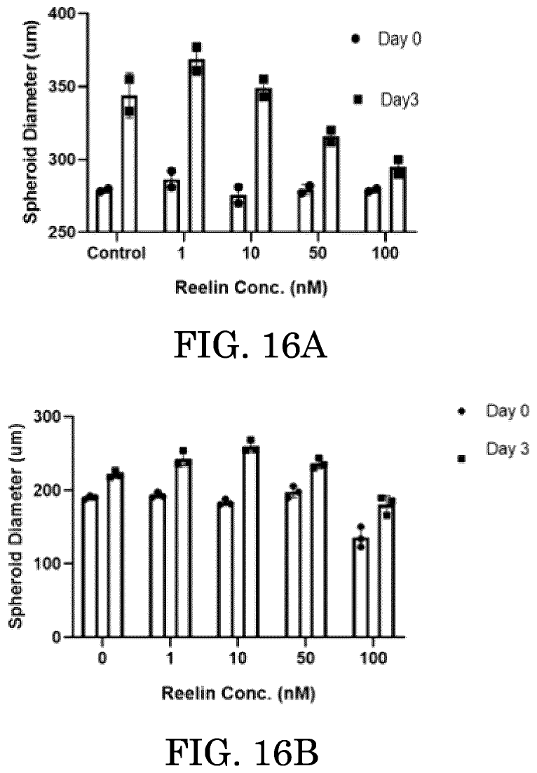

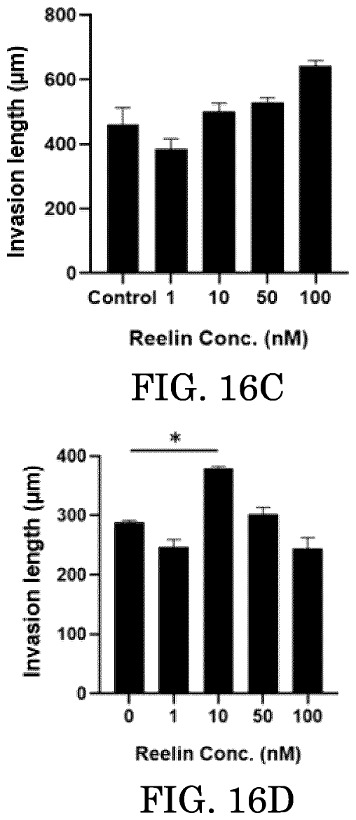

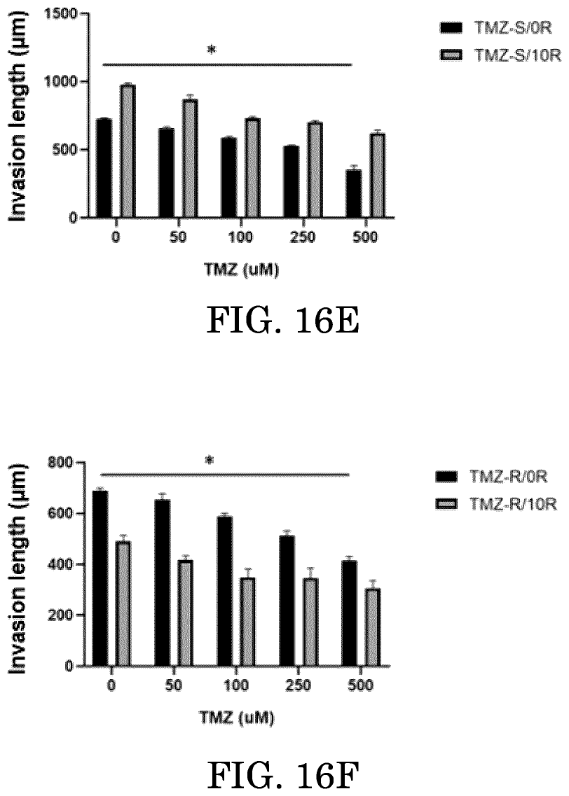

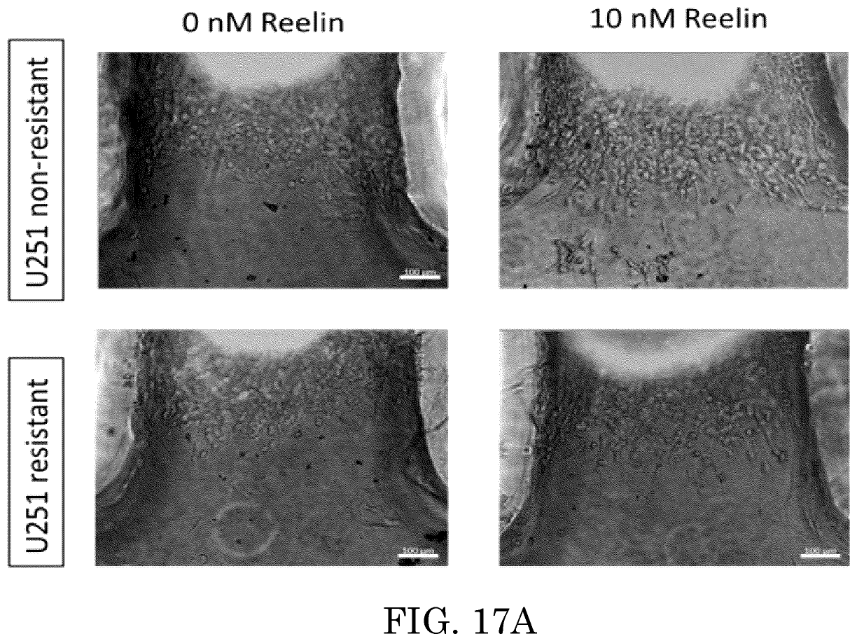

FIGS. 16A and B are graphs showing cell spheroids or organoids diameter versus Reelin concentration for non-resistant and resistant to Temozolomide (TMZ) cell spheroids or organoids respectively at day 0 and 3 of culturing in the device or sector. FIGS. 16 C and D show the invasion length of non-resistant and resistant to Temozolomide cell spheroids or organoids respectively within the collagen/HA ECM in the device or sector versus Reelin concentration. FIGS. 16 E and F show the invasion length non-resistant and resistant to Temozolomide cell spheroids or organoids respectively in Collagen/Reelin ECM versus TMZ concentration.

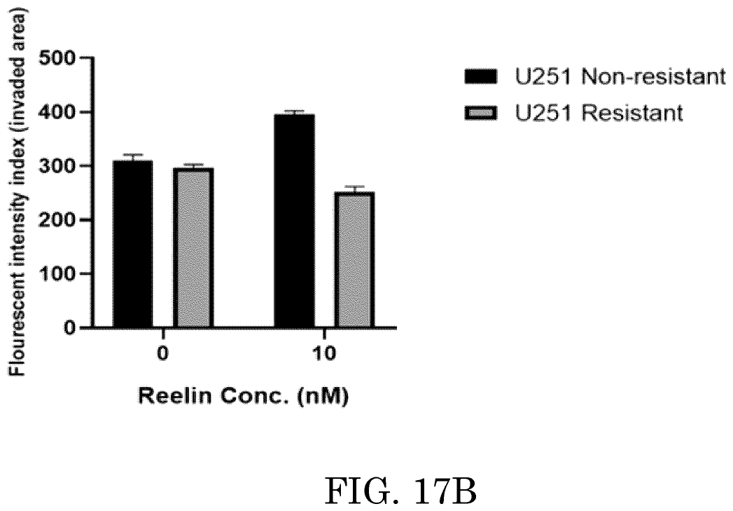

FIG. 17A demonstrates the immunofluorescent image of the invasiveness of the cell spheroids or organoids (resistant and non-resistant) with the collagen/HA ECM matrix at zero and 10 nM Reelin. Level of surface vimentin expression of the tumor cells was measured as an indication of invasiveness of cell spheroids or organoids with the ECM matrix. As depicted in FIG. 17B and in line of invasion length studies at FIGS. 16C and 16D, non-resistant cell spheroids or organoids showed higher fluorescent intensity index of the vimentin surface marker at 10 nm of Reelin in comparison to the resistant cell spheroids or organoids.

### Example 4

### Glioblastoma Cell Spheroids or Organoid Formation Using U251 Cells

The method of Glioblastoma cell spheroids or organoid formation was as described above.

Treatment of the Cell Spheroids or Organoids on with DFO in Normaxia and Hypoxia Conditions

Cell spheroids or organoids were treated on the device with Deferoxamine (DFO) drug to induce autophagy. DFO is an iron chelator drug with the capability of tumor cell killing through cell death induced autophagy. As a proof of concept, cell spheroids or organoids were treated with 50 μM of DFO for 3 days in normaxia and hypoxia conditions. Afterwards they were fixed with 10% formalin for staining with anti-HIF-1α and DAPI.

### Immunostaining of the Cell Spheroids or Organoids Using Slicing and Immunohistochemical (IHC) Analysis of the Cell Spheroids or Organoids in Hypoxia and Normaxia Conditions

IHC analysis of the cell spheroids or organoids after treatment was conducted followed by paraffin embedding and sectioning of the tumor tissues. In this regard, after fixation and washing of the cell spheroids or organoids in the device 10 or sector 20, 1% agarose solution was poured on top of the hydrogel insert to be embedded inside the agarose. The protocol was followed by serial dehydration of the tissue with 70, 80, 90 and 100% ethanol solution for 45 minutes. Afterwards, tissues were transferred into the Clearing Reagent (xylene) for 60 min. Tumor samples were finally prepared for paraffin blocking and sectioning with around 5 μm thickness. Tumor sections were de-paraffinized and re-hydrated using washing with 100 and 80 % ethanol and 3% hydrogen peroxide solution in methanol for 3 and 10 minutes respectively. Immunostaining of the tumor section was conducted following incubating the samples in Tris buffered saline (TBS)+0.3% Triton-X 100 (Permeabilisation) and 3% bovine serum albumin (BSA), 0.3% Triton X-100 in TBS (blocking) for 10 and 20 minutes respectively at room temperature. HIF-1α conjugated antibodies were used to observe the autophagy and hypoxia markers of the tumors in each tumor sections.

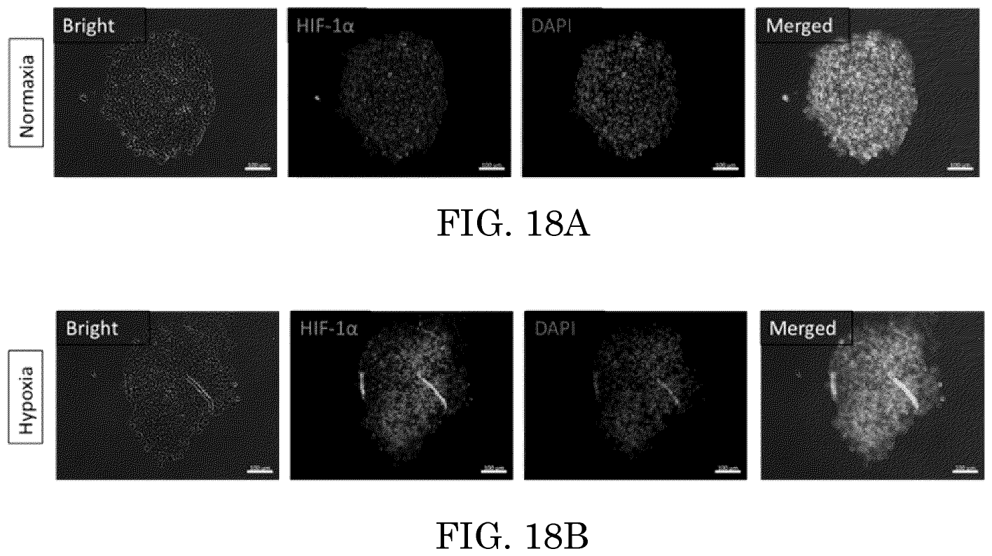

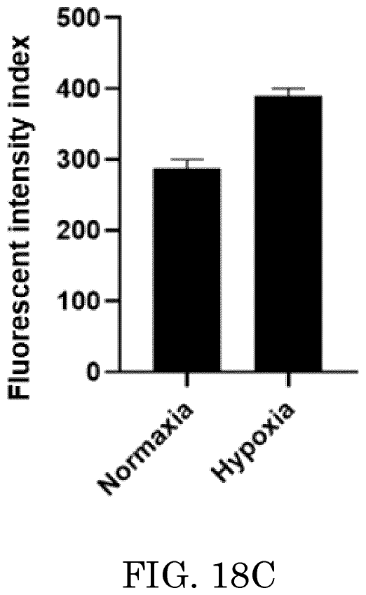

FIGS. 18A, B and C shows an increase in fluorescent intensity of the HIF-1 protein expression as a marker of hypoxic cells when treated with 50nM DFO in both normaxia and hypoxia conditions. FIGS. 18A and B shows immunohistochemistry fluorescence images of the U251 glioblastoma cell spheroids or organoid slices in A) normaxia and B) hypoxia conditions stained with HIF-1α and DAPI. FIG. 18C is a graph of fluorescent intensity of the HIF-1 α protein expression as a marker of hypoxic cells when treated with 50nM deferoxamine (DFO) in both normaxia and hypoxia conditions.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

### Example 5

### Pancreatic Co-Cultured Cell Spheroids Cancer Model Formation Using Panc-1 Cells and Fibroblast Cells Co-Seeding

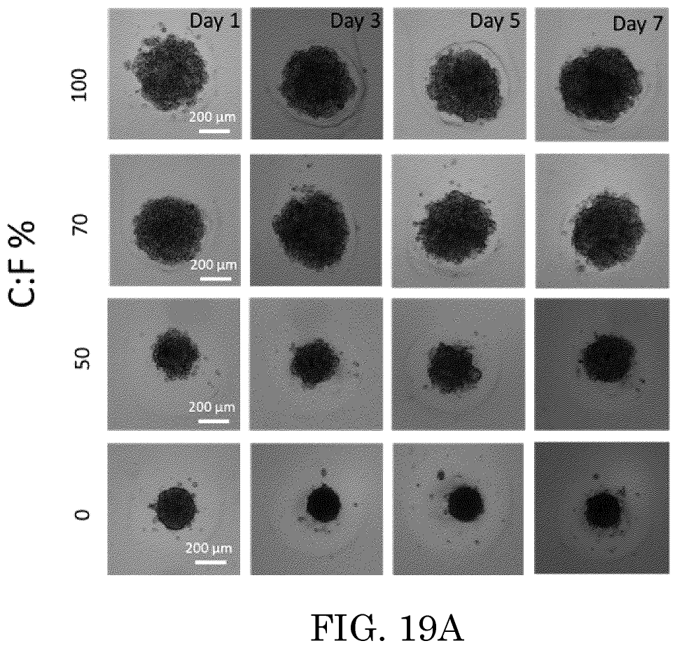

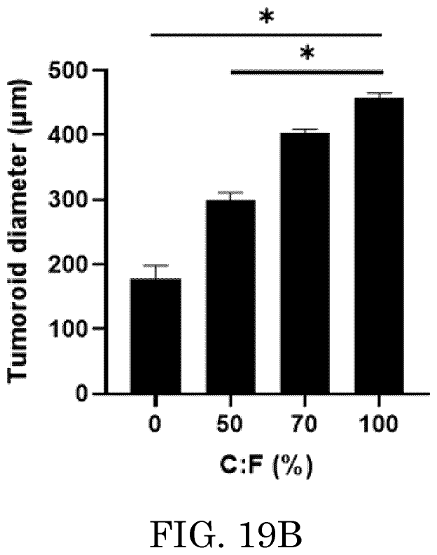

Pancreatic cancer cell line, Panc-1 were cultured in a culture medium consisting of Dulbecco's Modified Eagle Medium (DMEM) supplemented with 10% fetal bovine serum (FBS), and 100 mg/ml streptomycin. The Panc-1 cells were incubated at 37° C. in a humidified atmosphere of 5% CO2, and at 90% confluency, were trypsinized into a single cell suspension. The cell suspension was centrifuged at 300 rpm for 5 minutes to avoid dead cell sedimentation. After removing the supernatant, cells were suspended in 1 ml of medium and counted using a standard hemocytometer. To generate Panc-1 cell spheroid co-cultured model, Panc-1 cell suspension were mixed with the human-derived fibroblasts cell suspension (as the stromal component of the pancreatic tumor microenvironment) with the cancer to stromal cell ratio of 100, 70, 50 and 0. Cell suspension mixture was prepared in 50 μL of the culture medium and was applied gently through the loading well 12 of the device 10 or sector 20. The loaded device 10, 11, 13 or sector 20 was kept in an incubator for 10 minutes to let the cells fill the microwells 14 through the micro-troughs 16 of the device 10 or sector 20. Afterwards, the culture medium in the loading well 12 was aspirated and was exchanged with a new medium. 200 μL of the new medium was gently added to the seeded cell in the microwells 14 through the first delivery port 44. Panc-1/fibroblast co-cultured cell spheroids were monitored every day to measure their growth over the 4 days. FIG. 19 shows the growth and size changes of the co-cultured cancer spheroids in different cancer to stromal cell ratios.

### Invasion Study of the Co-Cultured Pancreatic Cancer Spheroids Model on the Device or Sector in the Presence of Collagen Hydrogel ECM

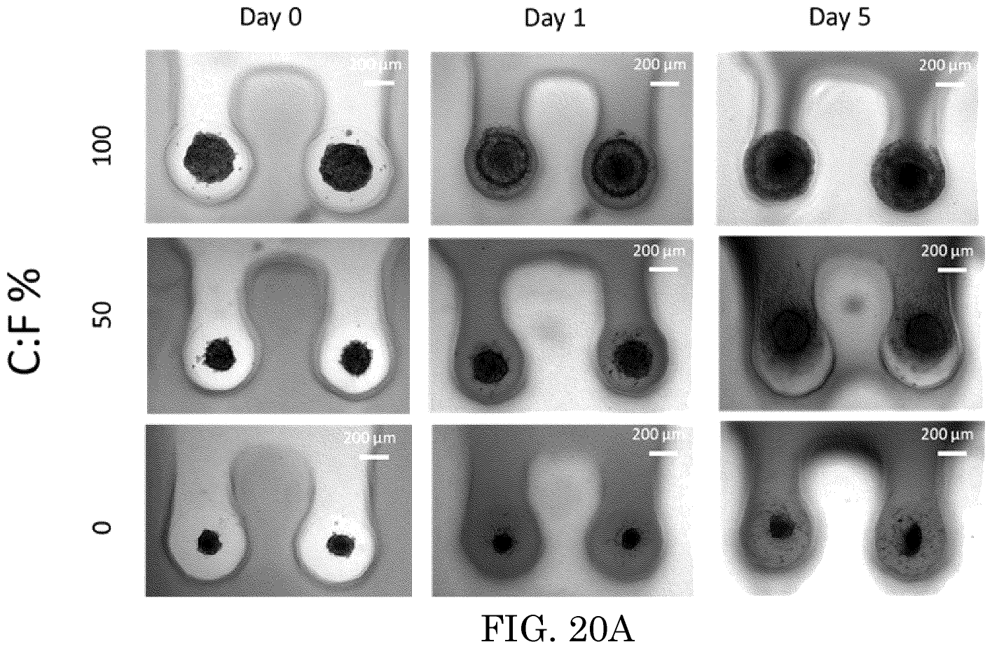

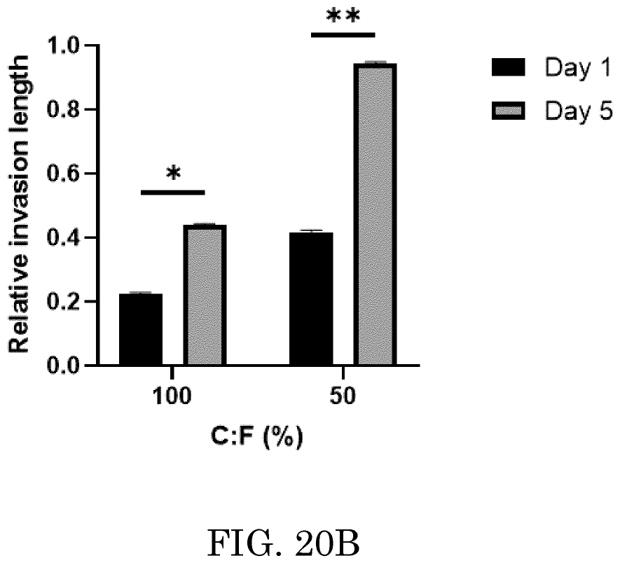

To investigate and compare the invasive behavior of co-cultured model with mono-cultured spheroids within the ECM hydrogel in device 10, or the sector 20, the cell spheroids or organoids were encapsulated in a collagen/hyaluronic acid hydrogel. The hydrogel formulation mimics the extra cellular matrix of the pancreatic tumor for better recapitulating of the tumor interaction with the environment. 50 μL of a hydrogel solution was gently pipetted through the loading well 12 of the device 10, 11, 13 or sector 20. It flowed through the micro-troughs 16 and filled the microwells 14 which retained the panc-1 cells. ECM hydrogel was formed after incubating at 37° C. for 30 min. FIG. 20 A shows the bright-field microscopic invasion images of the co-cultured cell spheroids in ECM hydrogel. FIG. 20B depicts the quantified invasion length of the co-cultured spheroids within the ECM.

### Pancreatic Co-Cultured Cell Spheroid Cancer Model Formation Using 2 Step of Panc-1 Spheroid Formation and Embedding in the ECM Laden Fibroblast Cells

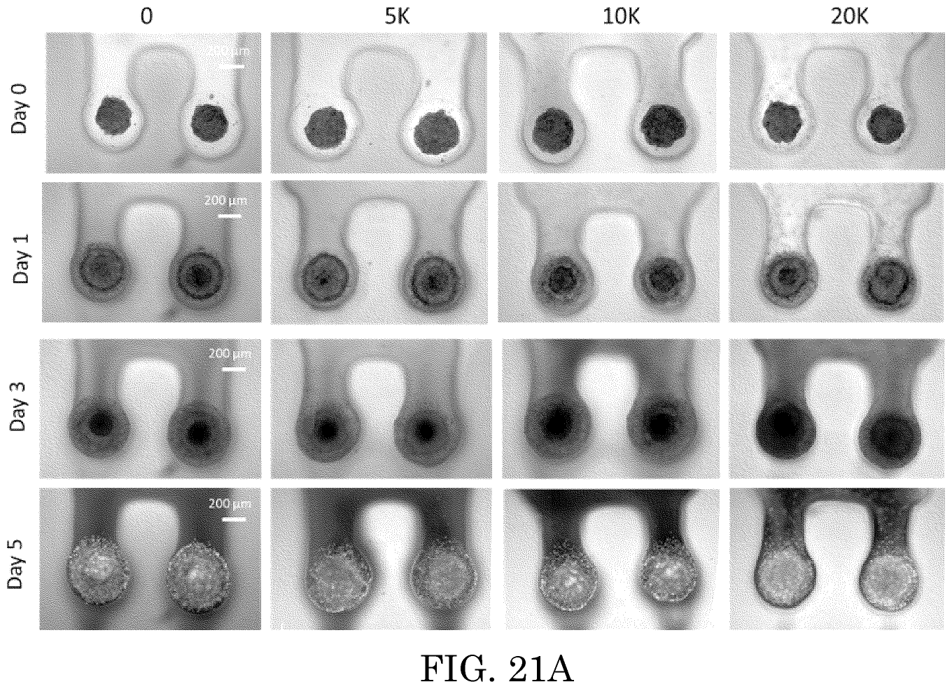

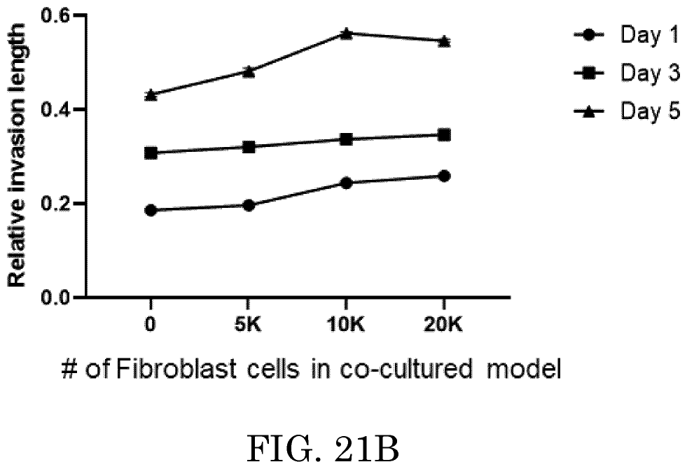

The method of pancreatic cancer cell spheroids formation was as described above. To investigate the effect of 2 step co-culture method on the invasive behavior of the cancer cells in device 10, or the sector 20, fibroblast cells with different numbers of 5000, 10,000, 20,000 were mixed with 40 μL of the ECM hydrogel and gently loaded through the first delivery port 44 or loading well 12 of the device 10, 11, 13 or sector 20. It flowed through the delivery trough 46 filled the microwells 14 which retained the panc-1 cells. ECM hydrogel was formed after incubating at 37° C. for 30 min. FIG. 21A i-iii shows the bright filed microscopic invasion images of the co-cultured spheroid model in ECM hydrogel in different days and different fibroblast number is each quadrant of the sector. FIG. 21B, depicts the quantified relative invasion length of the co-cultured spheroids with different conditions.

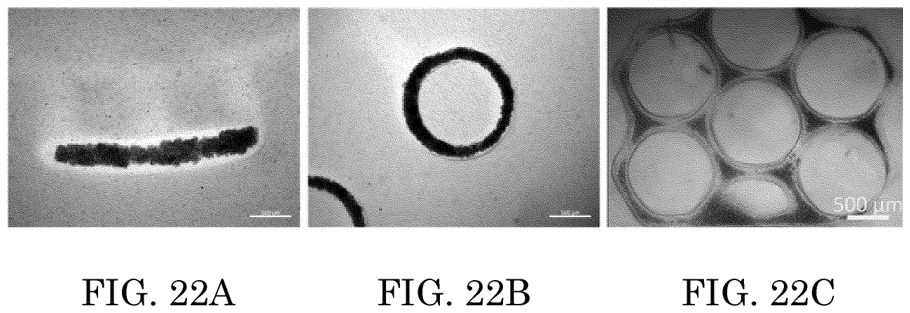

FIGS. 22A-C show cell aggregates of non-cancer cells (fibroblast cells cultured in the rod microwell, the ring microwell and the honeycomb microwell, respectively.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. A microfluidic device for use with a multi-well culture plate, for culturing cell spheroids or organoids for drug screening, the microfluidic device including at least one sector, each sector comprising: an outer edge; a plurality of microwells; a loading well which is in elevated relationship with the plurality of microwells; a plurality of micro-troughs which extend between the loading well and the plurality of microwells, such that each microwell is in fluid communication with the loading well via a micro-trough; a delivery port; and a plurality of delivery troughs which extend between the delivery port and the plurality of microwells such that each microwell is in fluid communication with the delivery port via a delivery trough.

2. The microfluidic device of claim 1, further comprising a multiplicity of segments, each segment defined by a center point and radially extending walls that radiate from the center point to the outer wall, the sector retained in the segment.

3. The microfluidic device of claim 2, each segment further comprising an inner wall disposed between the outer edge and the center point to define a second delivery port.

4. The microfluidic device of claim 3, each segment further comprising a second delivery trough which extends between the second delivery port and the microwell such that the second delivery port and each microwell are in fluid communication.

5. The microfluidic device of claim 4, wherein the sector is comprised of a biocompatible and transparent hydrogel.

6. The microfluidic device of claim 5, wherein each segment further comprises a pool which is disposed below the microwells.

7. The microfluidic device of claim 1 wherein the sector is comprised of a biocompatible rigid plastic polymer.

8. (canceled)

9. The microfluidic device of claim 1, wherein the radially extending walls are three-dimensionally printed in a well of the multi-well culture plate.

10. A microfluidic device for culturing cell spheroids or organoids and screening of therapeutics, the microfluidic device comprising: at least one sector, the sector including at least one cell spheroids or organoid forming module and a first delivery module, the first delivery module in fluid communication with the cell spheroids or organoid forming module; and an outer shell which retains the sector, wherein the outer shell includes a second delivery module which is in fluid communication with the cell spheroids or organoid forming module, the microfluidic device configured for retention in a well of a culture plate.

11. The microfluidic device of claim 10, wherein the cell spheroids or organoid forming module includes a plurality of microwells, a loading well which is in elevated relationship with the plurality of microwells, a plurality of micro-troughs which extend between the loading well and the plurality of microwells, such that each microwell is in fluid communication with the loading well via a micro-trough.

12. The microfluidic device of claim 11, wherein the first delivery module includes a delivery port, and a plurality of delivery troughs which extend between the delivery port and the plurality of microwells such that each microwell is in fluid communication with the delivery port via a delivery trough.

13. (canceled)

14. The microfluidic device of claim 12, wherein the second delivery module includes a second delivery port, and a delivery microchannel which extends between the second delivery port and the first delivery module such that the second deliver port is in fluid communication with the first delivery module.

15. The microfluidic device of claim 14, wherein the sector comprises at least one hydrogel.

16. The microfluidic device of claim 15 further comprising a pool, which is in indirect fluid communication with at least one microwell.

17. The microfluidic device of claim 16, wherein the pool is disposed below at least one microwell.

18. A method of screening at least two therapeutics, the method comprising: selecting a microfluidic device, the microfluidic device comprising at least one sector, the sector including at least one cell spheroids or organoid forming module and a first delivery module, the first delivery module in fluid communication with the cell spheroids or organoid forming module, the sector retained in an outer shell, the outer shell including a second delivery module, the second delivery module in fluid communication with the cell spheroids or organoid forming module; loading cells into the cell spheroids or organoid forming module; culturing the cells in the cell spheroids or organoid forming module to provide cell spheroids or organoids; loading a first therapeutic into the first delivery module; loading a second therapeutic into the second delivery module; and
determining the effect on cell spheroids or organoid function.

19. The method of claim 18, wherein the second therapeutic is loaded concomitantly with the first therapeutic.

20. (canceled)

21. (canceled)

22. (canceled)

23. A method of screening at least one therapeutic, the method comprising:
selecting the microfluidic device of claim 1; loading cells into the loading well;
culturing the cells in the microwells to provide cell spheroids or organoids; loading a first therapeutic into the delivery port; and determining the effect on cell spheroids or organoid function.

24. The method of claim 23 further comprising loading an ECM hydrogel into the loading well prior to loading the first therapeutic.

25. The method of claim 23, further comprising loading a second therapeutic into the delivery port after the first therapeutic is loaded.
